(19) United States
(12) Patent Application Publication
(10) Pub. No.: US 2014/0329322 A1
(43) Pub. Date: Nov. 6, 2014

(54) METHOD OF DIFFERENTIATING GLIOBLASTOMA CELLS

(71) Applicant: Allan Mishra, Menlo Park, CA (US)

(72) Inventor: Allan Mishra, Menlo Park, CA (US)

(21) Appl. No.: 14/332,194

(22) Filed: Jul. 15, 2014

Related U.S. Application Data
(62) Division of application No. 13/631,662, filed on Sep. 28, 2012.
Application No. 13/631,662 is a continuation-in-part of application No. 12/700,087, filed on Feb. 4, 2010, now abandoned.
Application No. 12/700,087 is a continuation of application No. 10/581,577, filed on Jun. 2, 2006, now Pat. No. 7,678,780.
(60) Provisional application No. 61/540,160, filed on Sep. 28, 2011.
(60) Provisional application No. 60/533,415, filed on Dec. 29, 2003.
(60) Provisional application No. 60/533,367, filed on Dec. 29, 2003.

(51) Int. Cl. C12N 5/09

(52) U.S. Cl. CPC C12N 5/0693
USPC 435/377

(57) ABSTRACT

A method of increasing differentiation of a cell line of glioblastoma cells is disclosed. A platelet-rich plasma (PRP) composition is prepared. The glioblastoma cells are cultured in the PRP composition for a time period sufficient for differentiation of the glioblastoma cells.

### CROSS-REFERENCE TO RELATED APPLICATIONS

This application is a divisional of U.S. application Ser. No. 13/631,662, filed Sep. 28, 2012, which is a continuation-in-part of U.S. application Ser. No. 12/700,087, filed Feb. 4, 2010, which is a continuation of U.S. application Ser. No. 10/581,577, filed Jun. 2, 2006, now U.S. Pat. No. 7,678,780, which is the U.S. National Phase of PCT/US2004/043088, filed Dec. 23, 2004 which claims priority to U.S. Provisional Application No. 60/533,415, filed on Dec. 29, 2003 and U.S. Provisional Application No. 60/533,367, filed on Dec. 29, 2003. This application also claims priority to U.S. Provisional Application No. 61/540,160, filed Sep. 28, 2011. The above disclosures are incorporated herein by reference.

### BACKGROUND OF THE INVENTION

1. Field of the Invention

This invention relates generally to the field of medicine and more particularly to formulations and methods of treating cancer.

2. Description of the Related Art

Treatment of cancer, wound healing, and a variety of chronic inflammatory diseases. is presently treated directly by physical means such as surgical removal of cancerous tissue, suturing of wounds and surgical removal of inflamed joints.

Further, each can be treated by chemical means. Chemotherapy is applied to cancers, growth hormones are applied to wound healing and anti-inflammatory drugs are applied to treating chronic inflammatory conditions. These, and related treatments are directed, in general, to treating the cancerous, injured, or inflamed tissue using active compounds not native to the patient's body. Embodiments of the present invention can be used along with all or any of these treatments.

However, in order to provide an understanding on how the present invention departs from conventional treatment modalities a brief and general description of current treatment technologies in these areas is provided.

### Cancer Treatments

The term “cancer” encompasses a spectrum of diseases that vary in treatment, prognosis, and curability. The approach to diagnosis and treatment depends on the site of tumor origin, the extent of spread, sites of involvement, the physiologic state of the patient, and prognosis. Once diagnosed, the tumor is usually “staged,” a process which involves using the techniques of surgery, physical examination, histopathology, imaging, and laboratory evaluation to define the extent of disease and to divide the cancer patient population into groups in order of decreasing probability of cure. Such systems are used both to plan treatment and determine the prognoses for the patient (Stockdale, F., 1996, “Principles of Cancer Patient Management,” In: Scientific American Medicine, vol. 3, Dale, D. C., and Federman, D. D. (eds.), Scientific American Press, New York).

The type or stage of the cancer can determine which of the three general types of treatment will be used: surgery, radiation therapy, and chemotherapy. An aggressive, combined modality treatment plan can also be chosen. To this end, surgery can be used to remove the primary tumor, and the remaining cells are treated with radiation therapy or chemotherapy (Rosenberg, S. A., 1985, “Combined-modality therapy of cancer: what is it and when does it work?” NewEngl. J. Med. 312:1512-14).

Surgery plays the central role in the diagnosis and treatment of cancer. In general, a surgical approach is required for biopsy, and surgery can be the definitive treatment for most patients with cancer. Surgery is also used to reduce tumor mass, to resect metastases, to resolve medical emergencies, to palliate and rehabilitate. Although the primary surgical technique for cancer treatment has involved the development of an operative field where tumors are resected under direct visualization, current techniques allow for some resections to be performed by endoscopic means. A primary concern in the treatment of cancer is the consideration of operative risk (Stockdale, F., supra).

Radiation therapy plays an important role in both the primary and palliative treatment of cancer. Both teletherapy (megavoltage radiation therapy) and brachytherapy (interstitial and intracavity radiation) are in common use. Electromagnetic radiation in the form of x-rays is most commonly used in teletherapy to treat common malignant tumors, while gamma rays, a form of electromagnetic radiation similar to x-rays but emitted by radioactive isotopes of radium, cobalt, and other elements, are also used. Radiation therapy transfers energy to tissues as discrete packets of energy, called photons that damage both malignant and normal tissues by producing ionization within cells. The target for the ions is most commonly the DNA; radiation therapy exploits the fact that the radiation damage is not uniform between malignant and non-malignant tissues—rapidly dividing cells are more sensitive to DNA damage than quiescent cells (Pass, H. I., 1993, “Photodynamic therapy in oncology: mechanisms and clinical use,” J. Natl. Cancer Instit. 85:443-56.) Radiation therapy is associated with unique benefits as well as important toxicities. Radiation is preferred in certain anatomic areas, (e.g., the mediastinum), where radiation may be the only feasible local method of treatment, and radiation may also be the only feasible local modality if tumor involvement is extensive. Radiation may also be used when the patient finds surgery unacceptable, or when the patient's medical condition prohibits a surgical procedure. Radiation treatment involves tissue damage which can lead to early and late radiation effects. The early effects (acute toxicity of radiation therapy) include erythema of the skin, desquamation, esophagitis, nausea, alopecia, and myelosuppression, while the late effects include tissue necrosis and fibrosis, and usually determine the limiting toxicity of radiation therapy (Stockdale, F., supra).

Nearly all chemotherapeutic agents currently in use interfere with DNA synthesis, with the provision of precursors for DNA and RNA synthesis, or with mitosis, and thus target proliferating cells (Stockdale, F., “Cancer growth and chemotherapy,” supra). Animal tumor investigation and human clinical trials have shown that drug combinations produce higher rates of objective response and longer survival than single agents (Frei, E. Ill, 1972, “Combination cancer therapy: presidential address,” Cancer Res. 32:2593-2607). Combination drug therapy uses the different mechanisms of action and cytotoxic potentials of multiple drugs, including the alkylating agents, antimetabolites, and antibiotics (Devita, V. T., et al., 1975, “Combination versus single agent chemotherapy: a review of the basis for selection of drug treatment of cancer,” Cancer 35:98-110). The physiologic condition of the patient, the growth characteristics of the tumor, the heterogeneity of the tumor cell population, and the multidrug resistance status of the tumor influence the efficacy of chemotherapy. Generally, chemotherapy is not targeted (although these techniques are being developed, e.g. Pastan, I. et al., 1986, “Immunotoxins,” Cell 47:641-648), and side effects such as bone marrow depression, gastroenteritis, nausea, alopecia, liver or lung damage, or sterility can result.

### Current Treatments—Immunology

The treatment regimens described above have had varying degrees of success. Because the success rate is far from perfect in many cases research continues to develop better treatments. One promising area of research relates to affecting the immune system. By the use of genetic engineering and/or chemical stimulation it is possible to modify and/or stimulate immune responses so that the body's own immune system treats the disease e.g., antibodies destroy cancer cells. This type of treatment departs from those described above in that it utilizes a biological process to fight a disease. However, the treatment is still a treatment that involves giving the patient an active compound not native to the patient.

### Cancer Cells

Neoplastic tissue and cancer arise in a myriad of forms. Cancer results from aberrant proliferation and differentiation coupled with invasion into normal tissue. Genetic alternations in cancerous cells result in many phenotypic changes. Cells no longer appear normal under a microscope. They have increased staining in their nuclei and at times severe alternations in cell shape, density and color. The surfaces of these cells are also quite different from normal tissue. Often, they have dramatically altered receptor patterns. Over expression or production of growth factor receptors are quite common. In this discussion Glioblastoma will be used as a specific example. Other types of neoplasia and cancer also have these issues and would likely be responsive to the same types of treatment.

Glioblastomas exhibit aberrant growth factor receptor expression on their cell surfaces. Autocrine signaling of various growth factors including platelet derived growth factor (PDGF) regulate glioblastoma survival. AVASTIN®, a monoclonal antibody that inhibits vascular endothelial growth factor (VEGF) has been approved for use against glioblastoma. Platelet rich plasma (PRP) contains a variety of powerful growth factors in superphysiologic concentrations including but not limited to epidermal growth factor (EGF), PDGF and VEGF. It is therefore counterintuitive to use PRP to treat Glioblastoma.

Recent studies reveal that aberrant constitutive activation of NF-κB (nuclear factor kappa-light-chain-enhancer of activated B cells), a latent transcription factor that acts as a suppressor of apoptosis, contributes to carcinogenesis and confers resistance to chemotherapy in a number of cancers. Furthermore, nuclear localization of p65, an indicator of NF-kB activation, was demonstrated in pathological specimens of surgically resected glioblastoma multiform (GBM) tumors. This is consistent with aberrant NFkB activation in glioblastomas. Without intending to be limited by theory, the growth factors within PRP may, via binding to one or more cell surface receptors, inactivate NFkB and therefore cause the cancer to change its behavior. Inhibition of NFkB by PRP releasate may also arise from known or unknown proteins within PRP.

Embodiments of the present invention can be utilized for treatments which involve a radical departure from normal treatments in that the present invention uses an active compound native to the patient being treated for affecting the cancerous, damaged or inflamed cells.

### SUMMARY OF EMBODIMENTS OF THE INVENTION

A method of treating cancer is disclosed whereby blood is extracted from a patient and platelets in the blood concentrated, e.g. to form platelet-rich plasma (PRP). The concentrated platelets are broken open in processing such as by subjecting them to ultrasound to break the platelets open and obtain platelet releasate. The releasate is formulated into an injectable formulation which is administered directly to the cancer e.g. injected into a tumor. A series of injections of a therapeutically effective amount of the formulation is repeatedly administered to the patient who may be the same patient from which the platelets were extracted. Particular components of the releasate may be concentrated or removed during the formation of the injectable formulation which may include the isolation of a single component or the inclusion of all the naturally occurring components but for a single component or components.

Embodiments of the invention are directed to formulations comprised of a patient's own platelet releasate.

Embodiments of the invention include methods whereby a patient is treated using an injectable formulation of specific molecules (e.g. individual growth factor or cytokine) isolated from the patient being treated.

Embodiments of the invention are directed to using a platelet releasate formulation as an adjunct in combination with one or more conventional cancer methodologies such as surgical removal of cancerous tissue, radiation and chemotherapy.

Embodiments of the invention are directed to methods of treating cancer by preparing a platelet-rich plasma composition from a patient in need of cancer treatment; formulating the platelet-rich plasma composition into an injectable formulation; and injecting the platelet-rich plasma composition into a patient in need of cancer treatment. In some embodiments, administering includes injecting the platelet-rich plasma composition into a tumor of the patient, preferably a cancerous tumor of the patient.

In some embodiments, the patient treated with the formulation is the same patient from which the blood is extracted from. Preferably, the platelet-rich plasma formulation is buffered to pH 7.4+/−5%.

In some embodiments, the platelet-rich plasma composition includes platelets in a concentration of about 500,000/μl to about 7,000,000 μl, monocytes in a concentration of about 400/μl to about 3200/μl, and neutrophils in a concentration of less than about 5000/μl. Preferably, the platelet concentration in the platelet-rich plasma composition is between 2-8 times the platelet concentration in the starting material such as whole blood or bone marrow aspirate, more preferably the platelet concentration in the platelet-rich plasma composition is between 4-6 times the platelet concentration in the whole blood or bone marrow aspirate.

In some preferred embodiments, the hemoglobin concentration in the platelet-rich plasma composition is less than about 3.5 grams per deciliter.

In some preferred embodiments, the platelet-rich plasma composition includes white blood cells. More preferably, the platelet-rich plasma composition has a ratio of lymphocytes+monocytes:neutrophils of at least 6:1, preferably at least 10:1, preferably at least 20:1 and more preferably at least 30:1.

In some preferred embodiments, the platelet-rich plasma composition includes neutrophils at a level of less than 1500 μl, preferably less than 1000/μl and more preferably less than 800/μl.

In some embodiments, the method also includes repeatedly injecting a therapeutically effective amount of the formulation to the patient over a period of time while monitoring the patient and adjusting dosing to effectively treat the cancer.

In some embodiments, injection of the injectable platelet composition is to an area where a tumor has been removed from the patient.

In preferred embodiments, the cancer is brain cancer, lung cancer, breast cancer, or colon cancer. Preferably, the cancer is brain cancer and more preferably, the brain cancer is a glioblastoma.

In some embodiments, the platelet-rich plasma composition does not contain an exogenous activator.

In some embodiments, the platelet-rich plasma composition is prepared from whole blood or bone marrow aspirate.

In some embodiments, the platelet-rich plasma composition is processed in a manner which breaks open the platelets to obtain an injectable platelet-rich plasma releasate for delivery to the cancerous tissue or area of cancerous tissue. Preferably, the processing includes exposing the platelet-rich plasma composition to energy waves.

In preferred embodiments, the patient treated with the formulation is the same patient from whom the blood is extracted from, and the formulation is buffered to pH 7.4+/−5%. More preferably, the platelets in the platelet-rich plasma composition are processed for a period of time and under conditions so as to break open 90% or more of the platelets.

In some embodiments, an exogenous platelet activator is administered to the patient.

These and other objects, advantages, and features of the invention will become apparent to those persons skilled in the art upon reading the details of the formulations and methods as more fully described below.

### BRIEF DESCRIPTION OF THE DRAWINGS

These and other feature of this invention will now be described with reference to the drawings of preferred embodiments which are intended to illustrate and not to limit the invention.

FIG. 1 is a graph of cell count versus time for cultured fibroblast cells in PRP.

FIG. 2 is a graph of cell count for three different concentrations of PRP releasate and a control.

FIG. 3 is a graph of cell counts over seven days for a control and a culture with sonicated PRP.

FIG. 4A shows glioblastoma multiforme cells after culture for 10 days with 10% (v/v) PRP. FIG. 4B shows glioblastoma multiforme cells after culture for 10 days without 10% (v/v) PRP.

### DETAILED DESCRIPTION

Before the present formulations and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a cell” includes a plurality of such cells and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “platelet” is used here to refer to a blood platelet. A platelet can be described as a minuscule protoplasmic disk occurring in vertebrate blood. Platelets play a role in blood clotting. The platelet may be derived from any source including a human blood supply, or the patient's own blood or bone marrow. Thus, the platelets in the composition of the inventions may be autologous. The platelets may be homologous, i.e. from a human but not the same human being treated with the composition.

The term “platelet-rich-plasma,” “PRP” and the like are used interchangeably here to mean a concentration of platelets in a carrier which concentration is above that of platelets normally found in blood (baseline). For definition purposes PRP is meant to be fraction of whole blood or bone marrow that contains an increased concentration of platelets when compared to baseline. For example, the platelet concentration may be 5 times, 10 times, 100 times or more the normal concentration in blood. The PRP may use the patient's own plasma as the carrier and the platelets may be present in the plasma at a range of from about 200,000 or less to 2,000,000 or more platelets per cubic centimeter. The PRP may be formed from whole blood e.g. by technology disclosed in any of U.S. Pat. Nos. 5,614,106; 5,580,465; 5,258,126 or publication cited in these patents and if needed stored by technology as taught in U.S. PGPUB 2002/0034722A1; U.S. Pat. No. 5,622,867 or publications cited therein. The “platelet-rich plasma composition” may include activated platelet-rich plasma, unactivated platelet-rich plasma, platelet releasate or a combination. The PRP may include blood components other than platelets. The PRP may be 50% or more, 75% or more, 80% or more, 95% or more, 99% or more platelets. The non-platelet components may be plasma, white blood cells and/or any blood component. PRP is formed from the concentration of platelets from whole blood, and may be obtained using autologous, allogenic, or pooled sources of platelets and/or plasma. PRP may be formed from a variety of animal sources, including human sources.

PRP may or may not contain white blood cells. In a preferred formulation, the PRP may contain increased platelets with increased white blood cells compared to baseline concentrations. In another preferred formulation, the PRP may contain increased platelets over baseline and increased white blood cells with the neutrophil component selectively reduced compared to baseline. A composition derived from whole blood or bone marrow that had been selectively depleted of neutrophils is preferred. A composition that contains predominately platelets, monocytes and lymphocytes could be obtained by selectively reducing the neutrophils from whole blood and or bone marrow aspirate. This last formulation also can be called neutrophil depleted PRP.

These compositions may be used in an activated or unactivated state or may be used as a platelet releasate. The platelet releasate may be obtained by using energy waves to release the contents of the platelets and other cells. These energy waves may be in the form of ultrasound or other types. The releasate may also be obtained by combining the PRP with thrombin and or calcium and then removing the supernatant after centrifugation. PRP may be prepared from an autologous source such as whole blood or bone marrow or it may be prepared from an allogenic source such as banked blood. The salient components of the PRP releasate may also be manufactured via genetic engineering techniques or others. Further applications and methods could include creating a PRP releasate and then storing it in a freezer (0 degrees C. to −80 degrees C.) to be used later or for multiple applications.

### Platelet Compositions

In accordance with the invention blood is extracted from a patient and platelets from the blood are concentrated. The platelets are processed in a manner so as to open the platelets and allow the platelet releasate to come out. The platelet releasate is then formulated such as by adding a buffering agent to adjust the pH to about 7.4±10% or ±5% or ±2% or ±1%. The processing of the platelets may be carried out by exposing the platelets to energy waves, agitation, chemical treatments, heat or other means so as to open the platelets and allow the releasate to come out. Preferably 90% or more of the platelets are opened, or 95% or more or substantially all of the platelets are opened. The platelet coverings may be removed or may become part of the releasate formulation which is buffered to a preferred pH range. The platelet releasate may be formulated with various salts in an aqueous injectable formulation which is administered to a patient. The patient is preferably the same patient from which the blood is extracted and the platelets are obtained.

When formulating the formulation it is possible to subject the formulation to various protein separation technologies including high pressure gas chromatography (HPGC) or high pressure liquid chromatography (HPLC) and the like or a variety of different protein separation technologies known to those skilled in the art. This can be done in order to separate out one or more of the growth factors, cytokines or other proteins present within the releasate. It is also possible to supplement the releasate by adding one or more proteins, growth factors, cytokines or other compounds to improve the therapeutic ability of the formulation. It is possible to separate away only a single growth factor, cytokine or protein. It is also possible to separate 2, 3 or any number of different components away from the platelet releasate. In addition, it is possible to add components which are not present or to supplement the percentage amount of proteins, growth factors and cytokines present with those which have been recombinantly produced. Thus, by combining different components in terms of growth factors, cytokines, proteins, etc. together a mixture can be tailored to treat the patient's particular cancer.

The PRP compositions generally comprise elevated concentrations of platelets and WBCs relative to whole blood. Typically, the concentration of RBCs and hemoglobin is depressed. Optionally, levels of neutrophils may also be depressed.

The PRP composition generally includes platelets at a platelet concentration that is higher than the baseline concentration of the platelets in whole blood. Baseline concentration means the concentration found in the patient's blood which would be the same as the concentration found in a blood sample from that patient without manipulation of the sample by laboratory technique such as cell sorting, centrifugation or filtration. The platelet concentration may be between about 1.1 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood contains at least 140,000 to 150,000 platelets per microliter and up to 400,000 to 500,000 platelets per microliter. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, bout 2,500,000 to about 5,000,000, or about 5,000,000 to about 7,000,000 platelets per microliter.

The WBC concentration is typically elevated in the PRP composition. For example, the WBC concentration may be between about 1.1 and about 2 times the baseline, about 2 and about 4 times the baseline, about 4 and about 6 times the baseline, about 6 and about 8 times the baseline, about 8 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000, about 10,000 and about 15,000, about 15,000 and about 20,000, about 20,000 and about 30,000, about 30,000 and about 50,000, about 50,000 and about 75,000 and about 75,000 and about 100,000.

Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher.

In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher.

The neutrophil concentration may additionally or alternatively be specified relative to the concentration of the lymphocytes and/or the monocytes. For example the ratio of monocytes+lymphocytes: neutrophils is preferably greater than 5, more preferably greater than 10, yet more preferably greater than 20, yet more preferably greater than 30, yet more preferably greater than 40, yet more preferably greater than 50, yet more preferably greater than 60, yet more preferably greater than 70, yet more preferably greater than 80, yet more preferably greater than 90.

One microliter of whole blood typically comprises 2,000 to 7,500 neutrophils. In some variations, the PRP composition may comprise neutrophils at a concentration of less than about 800 per microliter, preferably less than about 1,000 per microliter, more preferably less than about 1200 per microliter, more preferably less than about 1500 per microliter, yet more preferably less than about 2000 per microliter. Accordingly the neutrophils may be present at 500-2000 per microliter, more preferably at 800-1500 per microliter. In some embodiments, neutrophils may not be preferentially depleted and may be present at about 1,000 to about 5,000 per microliter, about 5,000 to about 20,000 per microliter, about 20,000 to about 40,000 per microliter, or about 40,000 to about 60,000 per microliter. Means to deplete blood products, such as PRP, of neutrophils is known as discussed in U.S. Pat. No. 7,462,268, which is incorporated herein by reference.

Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women.

In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl.

### Methods of Making

The PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the cancer, neoplasia, dysplasia, metaplasia or heteroplasia, the availability of prepared PRP, or any suitable combination of factors. Any suitable amount of blood may be collected. For example, about 20 cc to about 150 cc of blood may be drawn. More specifically, about 27 cc to about 110 cc or about 27 cc to about 55 cc of blood may be withdrawn. In some embodiments, the blood may be collected from a patient who may be presently suffering, or who has previously suffered from, cancer, neoplasia, dysplasia, metaplasia or heteroplasia. PRP made from a patient's own blood may significantly reduce the risk of adverse reactions or infection.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area.

The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume.

In some examples, the blood may then be centrifuged using a gravitational platelet system, such as the CELL FACTOR TECHNOLOGIES GPS SYSTEM® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

In accordance with the invention platelets may be concentrated from blood or bone marrow aspirate and sonicated to obtain a releasate and particularly components of the releasate isolated for use in treatments or isolated so that the remainder of the releasate is used in treatment. The white blood cells may be left in the releasate or removed prior to sonication. Further, the white blood cells may be isolated separately and sonicated as a whole or after sorting to isolate a particular class or type of white blood cell.

Platelets present a variety of antigens, including HLA and platelet-specific antigens. Patients transfused with platelets which are not their own often develop HLA antibodies. The patient may become refractory to all but HLA-matched platelets. When platelets are transfused to a patient with an antibody specific for an expressed antigen, the survival time of the transfused platelets may be markedly shortened. Nonimmune events may also contribute to reduced platelet survival. It is possible to distinguish immune from nonimmune platelet refractoriness by assessing platelet recovery soon after infusion, i.e., 10-60 minutes post-infusion platelet increment. In immune refractory states secondary to serologic incompatibility, there is poor recovery in the early post-infusion interval. In nonimmune mechanisms, i.e., splenomegaly, sepsis,—fever, intravascular devices, and DIG, platelet recovery within 1 hour of infusion may be adequate while long-term survival (i.e., 24-hour survival) is reduced. Serologic tests may be helpful in selecting platelets with acceptable survival. In accordance with the present invention the platelets are preferably taken from the same patient they will be used to treat. In a similar manner the platelet releasate or any portion thereof is taken from the same patient treated with the formulation. Alternatively, the patient is treated with platelets, platelet releasate and portions thereof extracted from a donor patient tested for and found to have a close serologic match with the patient being treated.

As the PRP composition comprises activated platelets, active agents within the platelets are released. These agents include, but are not limited to, cytokines (e.g., IL-1B, IL-6, TNF-A), chemokines (e.g., ENA-78 (CXCL5), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-β1, 2, and 3, and VEGF).

The PRP composition may be delivered as a liquid, a solid, a semi-solid (e.g., a gel,), an inhalable powder, or some combination thereof. When the PRP is delivered as a liquid, it may comprise a solution, an emulsion, a suspension, etc. A PRP semi-solid or gel may be prepared by adding a clotting agent (e.g., thrombin) to the PRP. The gel may be more viscous than a solution and therefore may better preserve its position once it is delivered to target tissue.

In some instances, it may be desirable to deliver the PRP composition as a liquid and have it gel or harden in situ. For example, the PRP compositions may include, for example, collagen, cyanoacrylate, adhesives that cure upon injection into tissue, liquids that solidify or gel after injection into tissue, suture material, agar, gelatin, light-activated dental composite, other dental composites, silk-elastin polymers, MATRIGEL® gelatinous protein mixture (Becton Dickinson Biosciences), hydrogels and/or other suitable biopolymers. Alternatively, the above mentioned agents need not form part of the PRP mixture. For example, the above mentioned agents may be delivered to the target tissue before or after the PRP has been delivered to the target tissue to cause the PRP to gel. In some embodiments, the PRP composition may harden or gel in response to one or more environmental or chemical factors such as temperature, pH, proteins, etc.

The PRP may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the PRP to physiological pH between about 6.5 and about 8.0. In certain embodiments, the physiological pH may be from about 7.3 to about 7.5, and may be about 7.4. For example, the buffering agent may be an 8.4% sodium bicarbonate solution. In these embodiments, for each cc of PRP isolated from whole blood, 0.05 cc of 8.4% sodium bicarbonate may be added. In some embodiments, the syringe may be gently shaken to mix the PRP and bicarbonate.

As noted above, the PRP composition may comprise one or more additional agents, diluents, solvents, or other ingredients. Examples of the additional agents include, but are not limited to, exogenous platelet activators such as thrombin, epinephrine, collagen, and calcium salts, pH adjusting agents, materials to promote degranulation or preserve platelets, additional growth factors or growth factor inhibitors, NSAIDS, steroids, anti-infective agents, and mixtures and combinations of the foregoing. In some embodiments, exogenous activators are specifically excluded from treatment.

Furthermore, the PRP compositions may comprise a contrast agent for detection by an imaging technique such as X-rays, magnetic resonance imaging (MRI), or ultrasound. Examples of such contrast agents include, but are not limited to, X-ray contrast (e.g., IsoVue), MRI contrast (e.g., gadolinium), and ultrasound contrast.

### Methods of Testing

In some variations, the PRP composition may be analyzed and/or modified prior to delivery to the patient. The PRP composition may be modified based on, for example, the condition to be treated, an initial complete blood count, a genetic profile of the patient, and other suitable factors.

In some embodiments, a patient's genetic profile is determined. The PRP composition of healthy individuals having the same or similar genetic profile is determined. A PRP composition is prepared in which components are matched to the PRP of the healthy individual having the same genetic profile. The modified PRP composition is administered to the patient to treat the disease or condition.

In some embodiments, the PRP composition of a patient, successfully recovering from a disease or condition may be used as a model to prepare a PRP composition to administer to a patient diagnosed with the same disease or condition. In other words, the PRP composition is first enriched in components which are effective in treating the disease based upon recovered or recovering individuals. The modified PRP composition is then administered to the patient suffering from the disease.

The PRP, or a portion of the PRP, may be placed into an automated blood analyzer that performs a compete blood count (CBC). As part of the CBC, the automated blood analyzers typically return a count of the number of platelets, WBCs, and RBCs present in the sample. The WBC count may further include counts of lymphocytes, monocytes, basophils, neutrophils, and/or eosinophils. Examples of blood analyzers that may be used include, but are not limited to, BECKMAN COULTER® LH series, SYSMEX XE-2100®, Siemens ADVIA® 120 & 2120, and the Abbott CELL-DYN® series.

It is believed that the effectiveness of treatments using PRP may be at least partially dependent on the genetic profile of the patient. The whole blood of a patient may be tested before and/or after generating the PRP composition to determine if the PRP composition is likely to be effective. Once the PRP has been determined to be useful, it may be delivered to the patient.

In certain variations, one or more genetic markers of a patient's DNA, mRNA, proteins, or the like may be evaluated prior to, during, and/or after delivery of the PRP composition. The patient's DNA, or other biomarkers, is typically captured via a sample such as blood, saliva, or other suitable body fluid or body tissue. The sample may be tested for genetic markers that are correlatable to the effectiveness of treatments using the PRP composition. In some instances, the identified genetic markers may be detectable using a genetic tool such as a gene chip or other genetic expression technology. In some instances, the genes that may be tested for include, but are not limited to, collagen type I (COL1A1), collagen type III (COL3A1), cartilage oligomeric matrix protein (COMP), matrix metalloproteinase-3 (MMP-3), and matrix metalloproteinase-13 (MMP-13). Such genetic tools can be used to measure changes in expression levels, or to detect single nucleotide polymorphisms (SNPs) which may be associated with a disease condition. Many gene chips are commercially available including the Affymetrix Gene Chip®, the Applied Microarrays CodeLink® arrays, and the Eppendorf DualChip & Silverquant®.

In some variations, the genetic tool may be analyzed to determine if the patient is likely to respond (favorably or unfavorably) to the PRP composition and/or to subsequent treatments. In certain variations, the PRP composition may be tested at a range of pH values and/or the pH of the PRP may be modified based at least in part on the genetic profile. In some instances, various genetic profiles may be associated with specific concentrations (or ranges of concentrations) as being more or less effective than other concentrations for various components of the PRP composition. The response to the PRP composition may be slowing or halting of cardiac apoptosis, anti-arrhythmia effects, or otherwise decrease risks associated with reperfusion therapy.

If the CBC returned by the automated blood analyzer is not within specified ranges, the PRP composition may be modified using a filtration device and/or cell sorter. The filtration device may use vacuum and/or gravity to remove a portion of the platelet, WBCs, and/or RBCs. In some variations, a cell sorter may receive a CBC input from an automated blood analyzer and/or a gene chip reader. A user may select or confirm one or more modifications to be made to the PRP composition. Of course, the cell sorter may be used with whole blood, portions of whole blood, and/or PRP. The cell sorter may sort the PRP composition based on electric charge, density, size, deformation, fluorescence, or the like. Examples of cell sorters include the BD FACSAria® cell sorter, the Cytopeia InFlux® cell sorter, those manufactured by Beckman Coulter, the Cytonome Gigasort® cell sorter, and the like.

Use of Platelet Rich Plasma compositions in drug discovery

Embodiments of the invention are directed to the use of platelet rich plasma compositions as described herein in drug discovery. A PRP composition is administered in a model system, preferably a mammalian model, such as a disease model for cancer or in the course of a human study. The effects of the administered PRP composition on cancer treatment and gene expression is monitored. For example, genes under- or over-expressed in successful treatments with PRP are identified as likely targets. Screening studies are conducted using the identified targets to identify molecules that may be effective for cancer treatment. For example, drugs, antibodies, proteins, metal salts, etc. having a similar effect are identified.

In a preferred embodiment, a PRP composition is used in a cancer disease model (in-vitro, animal, human, computer) to evaluate gene expression. Preferably, the disease model is a cell or tissue culture or an animal model or a human study. The PRP composition is used as a test treatment in the disease model compared to no treatment or other known treatments. DNA microarray, RNA, microRNA, epigenetics or other molecular analysis techniques are used to determine changes in gene expression due to the PRP treatment in the model. Cellular gene expression is evaluated and analyzed for patterns. Identified molecules are purified. Drugs are generated for treatment of the cancer and tested for efficacy. Specifically, enzymes, proteins or molecules that may be used to treat a specific disorder or condition are identified.

Fractionation of whole blood, PRP or its derivatives to identify known or unknown blockers of NFkB could be done. These blockers could then be used against neoplasia, cancer and specifically glioblastoma in an autologous form. Or, the specific blocking molecules could also be made via genetic engineering techniques and then these small molecules could be purified into drugs and then used against neoplasia in general, cancer or specifically glioblastoma. Genetic analysis of cell culture, animal or human trials could explore epigenetic markers, DNA microarray data, mRNA data, or microRNA data to identify other specific drug discovery targets that are producing this NFkB blockade. Novel drugs that block NFkB could then be made and tested against a variety of cancers including but not limited to glioblastoma.

In some embodiments, drug screening is specific for a patient or subpopulation of patients having a specific cancer. In some embodiments, a platelet rich plasma composition is administered to a patient suffering from a specific cancer and the effectiveness of the PRP composition is monitored in the patient. If the treatment is effective, a sample is taken from the patient, typically a bodily fluid such as blood or saliva or a tissue sample. The sample is analyzed for markers associated with recovery. The sample is used to determine the genetic profile of the patient using a DNA array (gene chip) or specific markers. This profile is compared to patients not responsive to the treatment. These may be diseased individual that did not respond to treatment with the PRP composition or healthy individuals. Based upon the difference in genetic profile, specific genes are identified as drug targets for the patient population responsive to the PRP composition. Other markers may be antigens, antibodies or small molecules. Drugs may be selected that mimic the effects of the PRP. Such drugs are candidates for disease treatment.

### Methods of Use

The PRP composition may be delivered at any suitable dose. In some embodiments, the dose may be between about 1 cc and about 3 cc, between about 3 cc and about 5 cc, between about 5 cc and about 10 cc, between about 10 cc and about 20 cc, or more. The dose may be delivered according to a medical procedure (e.g., at specific points in a procedure) and/or according to a schedule.

In some examples, the PRP composition may be delivered to cancer cells in situ. The PRP composition may be delivered to an individual in need thereof by injection using a syringe or catheter. The PRP composition may also be delivered via a dermal patch, a spray device or in combination with an ointment, bone graft, or drug. It may further be used as a coating on suture, stents, screws, plates, or some other implantable medical device. Finally, it may be used in conjunction with a bioresorbable drug or device.

In alternate embodiments, a platelet rich plasma composition is incorporated into an implantable medical device for timed release of PRP.

The site of delivery of the PRP composition is typically at or near the site of the cancer or neoplasm. The site of the cancer or neoplasm is determined by well-established methods including imaging studies. The preferred imaging study used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques.

The “dose” of platelets administered to a patient will vary over a wide range based on the age, weight, sex and condition of the patient as well as the patients' own normal platelet concentration, which as indicated above can vary over a tenfold or greater range. Doses of 1 million to 5 million platelets are typical but may be less or greater than such by a factor of two, five, ten or more.

The term “platelet releasate” is the PRP as defined above but treated so that what is inside the platelet shells is allowed to come out. The releasate may be subjected to processing whereby the platelet shells are removed and/or other blood components are removed or enriched, e.g. white blood cells, more specifically neutrophils, and/or red blood cells or remaining plasma is removed at least in part. The pH of the platelet releasate may be adjusted to physiological pH or higher or to about 7.4±10%, 7.4±5%, 7.4±2% or 7.4 to 7.6 as needed.

The terms “treatment”, “treating” and the like are used herein to generally mean obtaining a desired pharmacologic, physiologic or cosmetic effect. The effect may be prophylactic in terms of completely or partially preventing a condition, appearance, disease or symptom thereof and/or may be therapeutic in terms of a partial or complete cure for a condition and/or adverse effect attributable to a condition or disease. “Treatment” as used herein covers any treatment of a condition, disease or undesirable appearance in a mammal, particularly a human, and includes:
(a) preventing the disease (e.g. cancer), condition (pain) or appearance (e.g. visible tumors) from occurring in a subject which may be predisposed to such but has not yet been observed or diagnosed as having it;
(b) inhibiting the disease, condition or appearance, i.e., causing regression of condition or appearance.
(c) relieving the disease, condition or undesired appearance, i.e., causing regression of condition or appearance.
   The invention includes treating patients with platelet-rich plasma, activated or unactivated, and/or platelet releasate or components thereof formulated in accordance with the invention. Accordingly, the term “treatment’ is intended to mean providing a therapeutically detectable and beneficial effect of any kind on a patient.

The terms “synergistic”, “synergistic effect” and like are used herein to describe improved treatment effects obtained by combining one or more active components together in a composition or in a method of treatment. Although a synergistic effect in some field is meant an effect which is more than additive (e.g., 1+1=3) in the field of treating many diseases an additive (1+1=2) or less than additive (1+1=1.6) effect may be synergistic. For example, if one active ingredient removed 50% of a disease and a second active ingredient removed 50% of the disease the combined (and merely additional) effect would be 100% removal of the disease. However, the effect of both would not be expected to remove 100% of the disease.

Often, two active ingredients have no better or even worse results than either component by itself. If an additive effect could be obtained merely by combining treatments, then multiple ingredients could be applied to successfully treat any disease and such is not the case.

### Methodology/Radiation

Platelet releasate is shown to have an effect on cellular growth within the examples such as Examples 5 and 6 below. Those skilled in the art will understand that by taking tissue samples from the patient including tissue samples from a cancerous tumor it is possible to test different formulations on the tissue sample in order to determine the effect on the tissue.

In one aspect of the invention the patient is treated with a combination of the platelet releasate and conventional radiation therapy. More specifically, bone marrow cells are extracted from a patient that has been diagnosed as having cancer. Those bone marrow cells are placed on a cell culture medium which culture medium has been supplemented with platelet releasate or a portion of platelet releasate such as described above. The bone marrow cells are allowed to grow on the culture medium supplemented with the platelet releasate as described in detail within Example 7.

The patient from which the bone marrow cells were extracted is then subjected to intense radiation. The radiation treatments are intended to kill the patient's cancer cells. However, the radiation is sufficiently intense such that bone marrow cells of the patient are also destroyed making it substantially impossible for the patient to survive in the absence of new bone marrow cells. Accordingly, after the radiation has proceeded, and the cancer cells within the patient have been allowed to be confirmed as destroyed, the bone marrow cells which have been grown on the platelet releasate supplemented culture medium are formulated into an injectable formulation and readministered to the patient. Those bone marrow cells are allowed to grow in the patient. It is possible that the platelet releasate used to culture the bone marrow cells is platelet releasate obtained from the same patient. Alternatively, the releasate may be obtained from a healthy patient not suffering from cancer having been tested with various serological tests to provide the best possible match for the patient being treated.

### Methodology/Surgery

In accordance with the invention, surgery and formulations of the invention can be used together in the treatment of cancer. More specifically, blood is extracted from a patient and platelets are concentrated. The platelets are processed so as to obtain a releasate and the resulting releasate is formulated into an injectable formulation with the addition of a buffering agent in order to adjust the pH as indicated above. The platelets may be obtained from the patient suffering from cancer or may be obtained from a healthy patient which has been tested against the patient being treated in order to determine that a range of matches occur with respect to the patient's serological typing.

When an appropriate formulation comprising platelet-rich plasma, activated or unactivated and/or platelet releasate has been prepared a patient is subjected to a conventional surgery technique in order to surgically remove the cancerous tumor. After removal of the tumor the area where the tumor was removed from is treated with the activated or unactivated platelet-rich plasma and/or platelet releasate formulation of the invention. This is beneficial in a number of different ways. The platelet formulation can aid in improving wound healing. Further, the formulation can aid in modulating the inflammatory response. Lastly, the formulation can aid in modulating the growth of any cancer cells not removed surgically.

Thereafter, the patient may be repeatedly treated with the platelet formulation of the invention by periodically administering the formulation to the patient and, for example, specifically administering the formulation directly to the area for which the tumor was removed. The formulation may be a formulation from which the platelet shells are removed and/or from which one or more of the components of the platelet-rich plasma have been removed. Alternatively, the formulation may be supplemented with one or more pharmaceutically active components such as recombinantly produced growth factors or cytokines. In addition, the formulation may contain other small molecules such as anti-inflammatory agents, antibiotics, anesthetics and the like.

PRP in a variety of compositions and combinations could be used to treat neoplasia and cancer. One preferred composition of PRP would be one that contained increased concentrations of platelets compared to whole blood and or bone marrow with a white blood cell component that had been selectively depleted of neutrophils also known as granulocytes. Other forms of PRP including pure platelets in plasma and platelets with increased concentrations of unfractionated white blood cells could be used.

PRP could be used alone or in combination with other cancer treatments including but not limited to chemotherapy, radiation therapy, immunotherapy, stem cell therapy, cell therapy, gene therapy, gamma knife, surgery, electromagnetic therapy or a variety of other specific treatment protocols. The PRP could be given before during or after any or all of these therapies.

PRP could also be prepared to aid in the diagnosis and or tracking of cancer. Specific markers could be identified within PRP that are measured to evaluate a patient's status.

### Identification of Drug Targets to Treat Cancer

Embodiments of the invention are directed to methods of identifying drug candidates for treating a disease or condition based upon a response to a PRP composition, such as a PRP composition described above. In preferred embodiments, a PRP composition as described above is administered as a treatment to an individual suffering from a disease or condition. Alternatively, a model for the disease could be used such as an animal or cell culture system. The PRP composition is administered to the model animal or included in the cell culture media. In other embodiments, simulations are carried out using a computer. The efficacy of the treatment is monitored in the individual or animal model or in the cell culture or the computer simulation. Individuals responsive to the treatment are selected and a sample is obtained from the responsive individual. In the case of a human patient or animal model, this sample might be a bodily fluid sample such as blood or saliva or a tissue sample. In a cell culture, the responsive cells are selected. In a computer model, positive simulations are identified.

Analysis is performed on the biological sample obtained from the patient, animal or cell cultures. Typically such analysis would be by an immunoassay to determine the presence of specific antibodies or antigens or a genetic analysis. The genetic analysis may indicate genes that are upregulated or downregulated. In the case of a computer simulation, parameters are identified indicative of a positive response.

The results obtained as above are compared to results obtained from a non-disease population or a subpopulation having the disease but not responsive to treatment to determine targets present in the responsive population. Based upon the identified targets, drugs candidates, such as proteins or small molecules, for treating the disease condition are identified and further tested.

In preferred embodiments, the disease or condition is cancer. The cancer may be brain cancer, thyroid cancer, pancreatic cancer, liver cancer, breast cancer, or prostate cancer. The cancer may be leukemia, bladder cancer, cervical cancer, colon cancer, esophageal cancer, stomach cancer, skin cancer, or ovarian cancer.

### Evaluation of Proposed Treatment Plan

Smart computer systems could also be developed that analyze the value of using platelet rich plasma to treat cancer. These systems could also be used to evaluate other scientific or medical questions. Specifically, a search algorithm could be developed to analyze existing data in real time to determine if a specific hypothesis has value. This algorithm could be based on the overall number of pages dedicated to a particular topic, the links to that page and the time that page has been in existence.

To determine the value of a scientific paper or presentation a specific algorithm could be employed.

Paper's Scientific Value (PSV)=Number of Citations (C) divided by number of years (Y) since publication. If the paper has been published or presented less than one year a fraction of that year could be used. This could also be done for presentations as well as papers.

Specifically, if a paper has 200 citations and has been published for 10 years. The PSV would be 20. If the number of citations or references is 200 and the number of years since publication is 5. The PSV would be 40. The search engine would then place this paper above the paper with a PSV of 20.

Simply put: PSV=C/Y

Additionally, papers or presentations could be given an overall total based not only on the total number of citations but also on the quality of those citations. For example, each citation would have a value assigned to it based on its total citations and years since publication. This would be done on a running basis so that the value of paper or presentation would change based on its running citations.

Total Paper/Presentation Value (TPV) equals the PSV+

Each citation would have a PSV so the value of these citations could be incorporated into an overall estimate of value.

For example, Paper A could have 100 citations with the PSV of the citations equal to PSV1+PSV2, +PSV3 . . . PSV100. This total could be 500 with therefore an average of 5 citations per citation. This derivative value of citations (VC) could be multiplied or added to the initial PSV to get an overall value of the initial paper or presentation. The citations could also be divided by the number of years since publication.

For example, Paper A has 100 citations and was published 5 years ago. The average number of citations of these citations is 10. The PSV of Paper A equals 100 divided by 5 or 20. Then add 10 to get a total paper/presentation scientific value (TPSV) of 30.

Paper B has 100 citations and was published 5 years ago. The average number of citations of these citations is 5. The PSV of Paper B equals 100 divided by 5. Then add 5 to get a TPSV of 25. Paper A (30) is therefore more important than Paper B (25) and should be ranked higher by the search engine.

This simple algorithm could be employed alone or in combination with other algorithms to build a custom scientific search engine to help determine the value of using PRP for cancer or to answer any other question.

By adding in the social media component of a webpage in the context of a scientific question, another algorithm could be used alone or in combination with the one outlined above. Feeds from social networks such as Linkedin, Twitter, Facebook, Google+ and others could be combined to evaluate scientific or medical hypotheses or to rank or value information. These results could then be displayed on a search engine using a proprietary interface. The results could be combined with other algorithms or displayed alone.

The interface of the search engine could simply have two tabs, patients and professionals. The goal of the search engine would be to accelerate serendipity. This search engine could be free and supported by ads. The search engine could also be private and used only by paying for a subscription. The search engine could also be a social scientific search tool by invitation only. Scientists could be invited to submit specific sites or papers of value to be included in the search engine perhaps limited to a maximum of ten or twenty sites. Less or more may also be used. The collective group of invited or public could then vote on the value of each of these sites and or papers and then the algorithm could be recalculated on the basis of those votes.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

PRP was prepared using a centrifuge unit made by Harvest (Plymouth, Mass.). (Similar units are available as The Biomet GPS system, the Depuy Symphony machine and the Medtronic Magellan machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thrombin. The degranulated platelets are spun down and the releasate containing supernatant is recovered. The releasate may be optionally pH adjusted to a pH of 7.4 using sodium bicarbonate buffer.

### Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

### Cell Cultures of any Tissue

A researcher or clinician wishes to grow a cell culture of either fibroblasts or osteoarthritic cartilage cells. Using the technique of Example 1, an autologous platelet extract (APEX) is obtained and buffered to physiologic pH.

The cells are then isolated and grown in a media rich in the APEX in various conditions and dilutions. The APEX promotes cell differentiation and production of proteins such as collagen. The APEX may augment or promote the ability of the cells to transform into normal cells. Without intending to be limited by theory, it is hypothesized the APEX may convert the osteoarthritic cartilage cells to a more functional cell line that is reinjected into a diseased or injured joint. Alternatively, the APEX is directly introduced into an osteoarthritic joint to reverse the course of the disease. This is done under local anesthesia in a sterile manner.

### Example 5

### Human Fibroblast Proliferation in Buffered Platelet Rich Plasma

Platelet rich plasma has been used to augment bone grafting and to help accelerate or initiate wound healing. Fibroblasts are important components of the wound healing process. This example shows that human fibroblast cells will proliferate more in fetal bovine serum that has been augmented with a proprietary formulation of buffered platelet rich plasma.

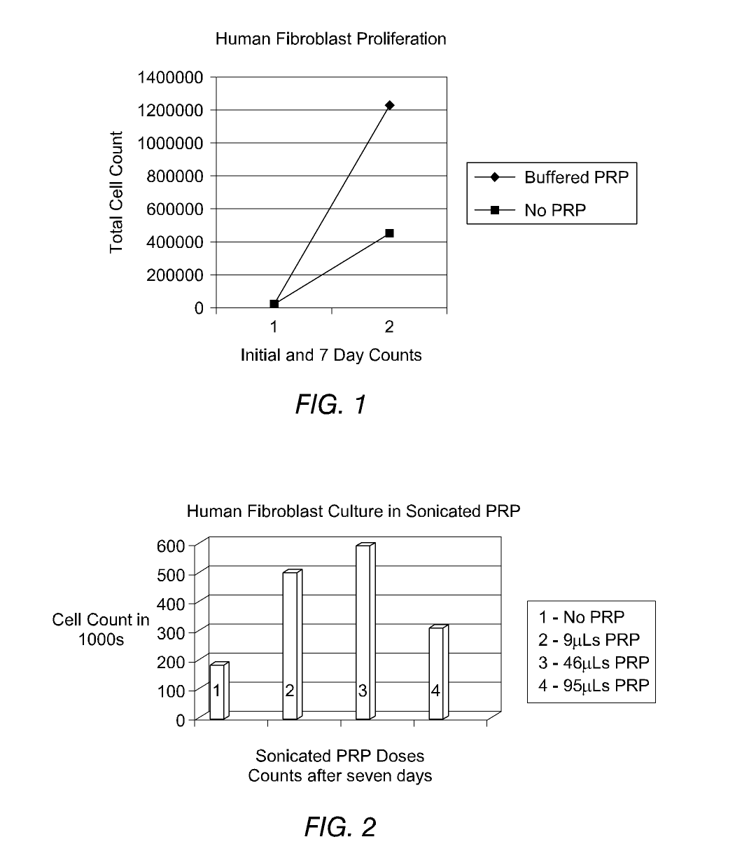

Human fibroblasts were isolated and then put into culture with 10% fetal bovine serum that had been augmented with a proprietary formulation of buffered platelet rich plasma (Group 1) or in 10% fetal bovine serum alone (Group 2). Initial cell counts were 25,000 in both groups. Seven days after initiating the culture experiment, the cells in each group were counted. The average total cell count in Group 1 (buffered PRP added) was 1,235,000. The average total cell count in Group 2 (No PRP) was 443,000. The group that was augmented with the buffered platelet rich plasma of the invention had 2.8 times the proliferation of the control group at seven days. (See FIG. 1)

Buffered platelet rich plasma augments human fibroblast proliferation when compared to the use of fetal bovine serum alone. This has significant implications for the use of buffered platelet rich plasma for either acute or chronic wound healing.

### Example 6

### Human Fibroblast Proliferation in Sonicated Platelet Rich Plasma

Human fibroblasts were isolated and then put into four different cultures. Three of the cultures comprised 10% fetal bovine serum that had been augmented with 9 uL, 46 uL, and 95 uL of buffered and sonicated platelet rich plasma. The fourth served as the control and was comprised of 10% fetal bovine serum. Initial cell counts were 20,000 in both groups. Variable doses of the sonicated PRP (sPRP) were seeded with cells.

Four days after initiating the culture experiment, the cells in each of the four groups were counted and the results are shown in FIG. 2. The cell count in the control group (No PRP) was 180,000 cells. The cell counts in the sonicated PRP group were as follows: 496,000 (9 uL dose of sPRP), 592,000 (46 uL dose of sPRP) and 303,000 (95 uL dose of sPRP).

This experiment shows that buffered, and sonicated platelet rich plasma augments human fibroblast proliferation when compared to the use of fetal bovine serum alone.

### Example 7

### Human Fibroblast Proliferation in Sonicated Platelet Rich Plasma

Human fibroblasts were isolated and then put into two different cultures. One of the cultures comprised 10% fetal bovine serum that had been augmented with buffered and sonicated platelet rich plasma. The other served as the control and was comprised of 10% fetal bovine serum. Initial cell counts were 20,000 in both groups.

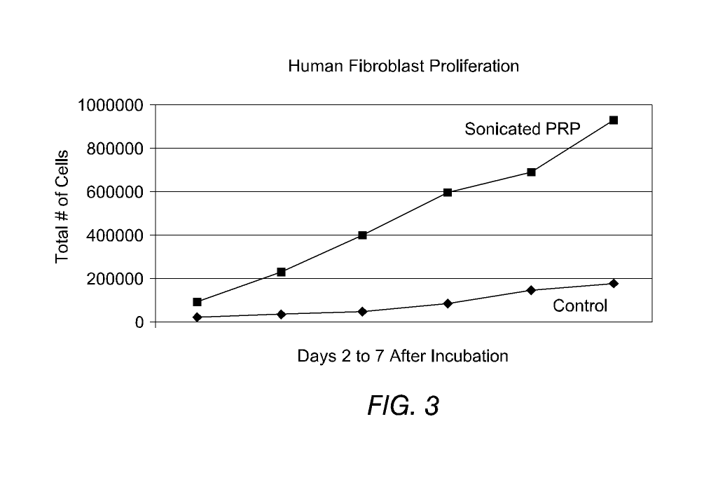

Seven days after initiating the culture experiment, the cells in each of the two groups were counted and the results are shown in FIG. 3. The cell count in the control group (No PRP) was 183,600 cells. The cell count in the sonicated PRP group was 924,800 cells. This experiment shows that buffered, and sonicated platelet rich plasma augments human fibroblast proliferation when compared to the use of fetal bovine serum alone. These results show the ability of the platelet releasate to promote cell growth and in particular fibroblast cells which are essential to firm, young looking skin.

### Example 8

### Culture of Bone Marrow Cells with PRP in Mice

Adult male and female CBA/J mice are obtained from a lab such as the Jackson Laboratory (Bar Harbor, Me.). All mice can be bred and maintained in an appropriate animal facility. Animals used may be 12 to 20 weeks old.

Bone marrow cells are collected by flushing the tibias and femurs of CBA/J mice with modified Dulbecco's phosphate-buffered saline (PBS) using a sterile syringe and 25-gauge needle. Homogenous single-cell suspensions are obtained by the repeated passage of cell mixtures through a Pasteur pipet. All cells are washed twice by centrifugation at 250 g for 10 min in PBS and then assessed for viability by trypan blue dye exclusion. Cells are then adjusted to the desired concentration prior to use. Bone marrow cells (250,000) are cultured in 96-well round-bottom microtiter plates, e.g. (Flow Laboratories, Mississauga, Ontario, Canada). The culture medium may be serum-free RPMIplus 4 mM L-glutamine, 20 mM Hepes, 100 U/ml penicillin, 100 μg/ml streptomycin (GIBCO Laboratories, Burlington, Ontario, Canada), 5 μg/ml transferrin, and 5×10⁵ 2-mercaptoethanol (Eastman Chemicals Co., Rochester N.Y.). Cells are cultured in the presence or absence of PRP and/or releasate at a concentration of 400 μg/ml, respectively. Total volume of all cultures may be 0.2 ml. Cultures are maintained at 37° C. in 95% humidified air and 5% CO₂. Six hours prior to harvesting, the cultures are pulsed with 1 μCi tritiated thymidine (NEN, sp act 77.1 Ci/mmol). Cells are then harvested on glass fiber mats (Flow Labs) with a multiple sample harvester (Skatron, Flow Labs). Water-insoluble tritiated thymidine incorporation is measured with an LKB 1215 Rackbeta II using standard liquid scintillation techniques.

The effects of PRP on cultured murine bone marrow may be evaluated in serum-free medium. In this experiment, 2.5×10⁵ viable cells from bone marrow of CBA/J mice may be cultured for 72 hours in serum-free RPMI media in the presence or absence of PRP at a final concentration of 400 μg/ml and transferrin at a final concentration of 5 μg/ml.

As demonstrated in Examples 5 and 6 PRP and releasate are each effective in promoting the proliferation of cells and accordingly useful for therapy involving the promotion of cell proliferation. This suggests it is useful in the proliferation of bone marrow cells, which would be useful in the treatment for the prevention of side effects of immunosuppressive therapy, radiotherapy or chemotherapy, or other therapies known to depress the immune system and suppress bone marrow production, causing myelotoxicity. Accordingly, PRP and/or releasate is employed to treat deficiencies in hematopoietic progenitor or stem cells, or related disorders.

PRP and/or platelet releasate may also be employed in methods for treating cancer and other pathological states resulting in myelotoxicity, exposure to radiation or drugs, and including for example, leukopenia, bacterial and viral infections, anemia, B cell or T cell deficiencies, including immune cell or hematopoietic cell deficiency following autologous or non-autologous bone marrow transplantation. PRP and/or platelet releasate may also be employed to stimulate development of megakaryocytes and natural killer cells in vitro or in vivo.

The media, compositions, and methods of the invention are also useful for treating cancers that are treated by bone marrow transplants (BMT) that involve removing bone marrow cells from the patient, maintaining these cells in an ex vivo culture while the patient is treated with radiation or chemotherapy, and then transplanting these cells back into the patient after the treatment has been completed to restore the patient's bone marrow. Accordingly, PRP and/or platelet releasate may be employed for BMT as a means for reconstituting bone marrow in ex vivo cell culture medium and for promoting bone marrow cell proliferation in vivo. PRP and/or platelet releasate is also useful for other cell therapies, e.g. cell expansion and/or gene therapy protocols, therapies requiring ex vivo cell culture. PRP and/or platelet releasate is also useful in the prevention of autologous or allogenic bone marrow transplant rejection.

### Example 9

### Culture of Glioblastoma Cells in the Presence of PRP

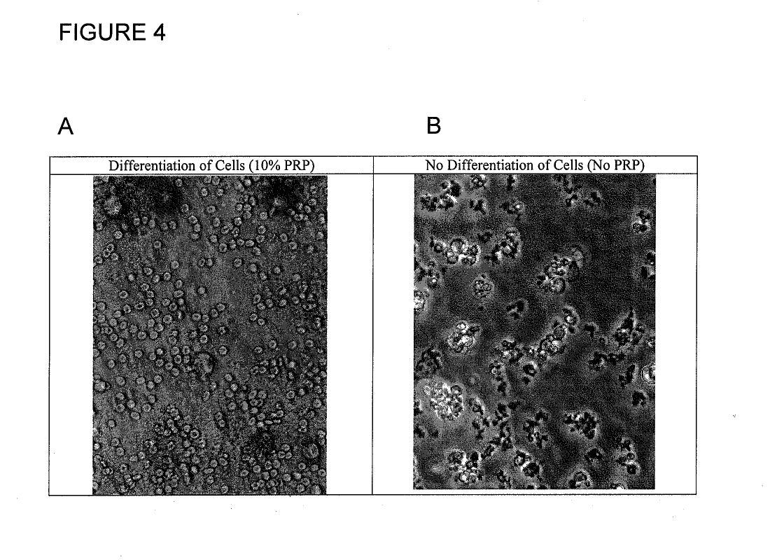

Platelet rich plasma was prepared by drawing 55 cc of whole blood from a peripheral vein into a syringe containing 5 cc of ACD as an anticoagulant. Using a device that separates blood into its components via centrifugation, PRP was prepared. This type of PRP contained platelets approximately 4-5 times baseline with increased concentrations of white blood cells compared to baseline. After preparation, sodium bicarbonate was used to titrate the preparation to a physiologic pH in the range of 7.3 to 7.5. Other ranges may also be used as deemed appropriate including below 7.3 and above 7.5. The composition was then subjected to ultrasonic waves at 5 watts for 8 seconds. This produced a platelet rich plasma releasate. PRP was then used at 10% concentration by volume as a culture media with Glioblastoma Multiforme cells for 10 days and compared to standard culture media without PRP. At the end of that time period, marked phenotypic differences in the cell lines were noted. The culture treated with 10% PRP had cell morphology consistent with differentiation of the cell line compared to the untreated cell line that continued to exhibit anaplastic cancerous features. See FIGS. 4A (with 10% PRP) and 4B (control). There are clear phenotypic differences between the cell lines that point to the value of using PRP as a treatment.

### Example 10

### Treatment of Brain Tumor with PRP

A patient presents with a diagnosis of a benign or malignant brain tumor. This patient then elects to undergo surgery to remove the brain tumor. The tumor is then resected surgically. Optionally, the tumor is tested for a variety of genetic markers including but not limited to DNA, mRNA, cell surface markers, microRNA or other to determine if platelet rich plasma (PRP) would be an effective primary or adjuvant treatment for the tumor. This step may be skipped altogether especially if data already exists to support the use of PRP for this tumor type.

After tumor resection, the surgeon may apply PRP to the bed of tumor, inject it into the surrounding tissue or combine PRP with a variety of carrier agents such as a hydrogel, suture, collagen sponge or other implantable device to the immediate tumor area. Subsequent to that application, the patient may undergo further treatment in the form of chemotherapy, radiation therapy or other types of cancer treatments including but not limited to gene therapy, immunotherapy or cell therapy. The order of these treatments may also be changed, for example, with the patient receiving any or all of these therapies then having the tumor resected and then treated with PRP. Finally, the tumor may be treated with PRP prior to surgical resection. It should be known to those practiced in the art that the application of the PRP to the tumor could be done via a variety of methods including but not limited to injection directly into the tumor, intravenous, intrathecal or intra-arterial infusion and be guided by a variety of imaging modalities including but not limited to x-ray, CT, MRI, PET scan or others.

### Example 11

### Treatment of Intracranial Tumor with PRP

A patient presents with an intracranial tumor. The clinician would then treat this tumor with platelet rich plasma in a releasate form or other form (unactivated without forming a releasate) via the following protocol.

The tumor is mapped via MRI, CT, PET scan or other imaging modality. The patient is then taken to a procedure room. Sedation may or may not be given.

The PRP is prepared via the patient's own blood or could be made from an allogenic source. A catheter is inserted into a vein, artery or into the spinal canal or even directly into the brain via a drill hole. Fluoroscopic or other guidance could be used to guide the PRP appropriately to the tumor location. Mannitol may or may not be used to open up the blood brain barrier. The PRP releasate is then injected via a syringe or catheter or other device into the tumor bed via an intravenous, intra-arterial or intrathecal pathway. Specialized catheters could be used to penetrate into the tumor itself or tumor bed and then deliver the PRP.

### Example 12

The methods of example 11 could be used alone or in combination with surgery, chemotherapy agents, radiation therapy, immunotherapy, gene therapy, cell therapy (stem cell adult or embryonic as one example) or other treatment modalities. These treatments could be given before and/or after treatment with PRP. One specific example would be to resect the tumor surgically, treat the tumor bed immediately with PRP releasate and then radiate the tumor post operatively. Adding other chemotherapeutic agents to this regimen may also be an option. Repeating the PRP treatment and then repeating radiation and or chemotherapy may be another option.

These methods could be used to treat a tumor in any location of the body including but not limited to the brain, lung, breast, internal organ (pancreas, liver, etc.) or to treat a primary or metastatic cancer of the musculoskeletal system such as a sarcoma.

### Example 13

PRP is prepared and co-cultured with glioblastoma cells (or other cancer cells). DNA microarray techniques or other techniques such as microRNA, mRNA measurement or evaluation of epigenetic markers are used to evaluate upregulated genes or pathways. Analysis of the data identifies drug discovery targets. PRP may also be prepared and then analyzed for unique inhibitors or enhancers of the NFkB pathway. Novel molecules or pathways may be identified and then used to treat glioblastoma or other cancers.

The use of the PRP could be used to treat cancer in an unactivated form, an activated form or in the form of a releasate prepared by a variety of methods including but not limited to gravity, centrifugation, cell sorting and others. Ultrasound or other energy waves may be used to obtain the releasate.

### Example 14

### Use of PRP in Drug Discovery for Cancer Treatments

A treatment employing a PRP composition is used in a cancer trial in an in-vitro, animal or human model. For example, PRP could be injected into or around a tumor. The effects of the treatment on gene expression is determined using microarrays. Computer analysis of microarray output is done to seek out specific upregulation or downregulation of markers of apoptosis, cell regulation or any new or existing signaling pathways. Based on genetic expression in successfully treated individuals, genes are identified which are upregulated or downregulated in response to effective treatment. Drugs affecting the identified genes are used for the treatment of cancers of any or all types including but not limited to brain cancer, lung cancer, breast cancer, colon cancer or other neoplastic disorders.

While the described embodiment represents the preferred embodiment of the present invention, it is to be understood that modifications will occur to those skilled in the art without departing from the spirit of the invention. The scope of the invention is therefore to be determined solely by the appended claims.

What is claimed is:

1. A method of increasing differentiation of a cell line of glioblastoma cells comprising:
preparing a platelet-rich plasma (PRP) composition; and
culturing glioblastoma cells in the PRP composition for a time period sufficient for differentiation of the glioblastoma cells, wherein the PRP composition does not contain an exogenous activator.

2. The method of claim 1, wherein the formulation is buffered to pH 7.4+/−5%.

3. The method of claim 1, wherein the PRP composition is prepared from whole blood or bone marrow aspirate.

4. The method of claim 1, wherein preparing the PRP composition comprises processing the PRP composition in a manner which breaks open the platelets, thereby obtaining a PRP releasate.

5. The method of claim 4, wherein the processing comprises exposing the PRP composition to energy waves.

6. The method of claim 5, wherein the PRP composition is sonicated.
